(19) United States
(12) Patent Application Publication
(10) Pub. No.: US 2011/0135577 A1
(43) Pub. Date: Jun. 9, 2011

(54) Superparamagnetic nanoparticles IN MEDICAL THERAPEUTICS and manufacturing method THEREOF

(75) Inventors: Hsi Chin Wu, Taipei (TW); Feng Huei Lin, Taipei (TW); Tzu Wei Wang, Hsinchu (TW)

(73) Assignee: National Taiwan University, Taipei City (TW)

(21) Appl. No.: 12/630,485

(22) Filed: Dec. 3, 2009

(51) Int. Cl. A61K 49/18; A61K 9/50; A61K 31/7088; A61K 38/16; A61K 38/02; A61K 39/395; A61K 31/20; A61K 31/7105; A61K 31/711; A61P 35/00; A61P 43/00

(52) U.S. Cl. 424/9.32; 424/490; 514/44 R; 424/130.1; 514/558; 424/9.1; 514/1.1; 514/19.3

(57) ABSTRACT

The successful transfer of therapeutic agents such as genetic materials (e.g. nucleic acid) or drug into living cells is the most important issue depending on the development of the delivery carrier. A method for manufacturing superparamagnetic nanoparticles in medical therapeutics is described to develop nano-sized calcium phosphate (CaP) mineral was rendered magnetic as delivery vehicle. The CaP-based magnetized nanoparticles (NPs) were possessed superparamagnetic property by hetero-epitaxial growth of magnetite on the CaP crystallites and also showed no harm to the cultured cells and elicited no cytotoxicity. The magnetized CaP was demonstrated to have good plasmid DNA binding affinity or drug carrying capacity. It significantly increased the expression of gene transfection and efficiency in delivery to mesenchymal stem cells (MSCs) under exogenous magnetic field. According to the above facts, this newly-synthesized magnetized CaP NPs has great potential as a novel non-viral targeted delivery vehicle to be applied for medical applications.

### BACKGROUND OF THE INVENTION

1. Field of Invention

The present invention relates to genetic material delivery vehicle, especially nanoparticles (NPs) with superparamagnetic property and a manufacturing method thereof. The present invention is further related to in vitro and in vivo targeting therapeutic agent for disease or signal mediator for tissue regeneration in biomedical applications.

2. Related Art

With the decoding of gene sequence and improvement of molecular biotechnology, gene therapy has become a novel treatment manner in wide variety of diseases of both genetic and acquired origin such as cancer, inherited monogenic disorders, neurologic disease and so on. The outcome from these trials has established gene therapy with great potential to revolutionize the treatment of human disease. However, the success of gene therapy is particularly dependent on the development of the gene delivery carrier.

Generally, two different types of delivery system are used: viral and non-viral. Viral infection is the traditional method for gene transfer based on adenovirus, retrovirus and others. It has a great performance to transfer deoxyribonucleic acid (DNA) into cells and nearly 80% of the recent clinical protocols use this method. However, it has potential drawbacks and limitations, such as the risk of carcinogenity, immunogenicity, restricted DNA carrying capacity and high cost. Many limitations and potential clinical risks of viral vectors have led to the direction shift to focus towards non-viral delivery systems. Non-viral gene delivery system is safer with less pathogenicity concerns. Currently, gene transfection into target cells using naked DNA has been improved by combining with several physical techniques; for example, electroporation, gene gun, ultrasound and hydrodynamic pressure. Chemical approaches are also used such as NPs including lipid, polymer and inorganic composite. These have been utilized to improve the efficiency and specificity of gene transfer. So far, low efficiency is the major problem of non-viral system.

While the concept of NPs in drug delivery is not new, technological advances have accelerated recent development of NPs as delivery vehicles for a wide array of therapeutic agents. NPs have undergone investigation for myriad applications—including treatment of tumors by delivery of anticancer agents, and treatment of focal degenerative conditions, and enhancement of tissue repair/regeneration through delivery of selected proteins and their encoding genes, and regulating RNA.

Several inorganic NPs have been investigated for cellular delivery due to well-established manufacturing process. Moreover, many advantages of inorganic NPs show microbial attack escapement, easy preparation, low toxicity, and satisfactory storage stability over organic NPs. Using calcium phosphate as a gene delivery vector and transfection tool was originally discovered by F. L. Graham and A. J. van der Eb in 1973. It is known that calcium phosphate is the main component in hard tissue of human body. It possesses good biocompatibility, adequate biodegradability and non-toxicity properties suitable as a biomaterial candidate. Using calcium phosphate is still a straightforward transfection method compared to other NPs according to its superior biocompatible properties. However, the irreproducibility and low efficiency are the most concerned issues due to the parameters of the calcium phosphate precipitation method. Challenges in employing calcium phosphate for the delivery of therapeutic agents include their targeting of, and their retention at, a specific site.

The series of magnetic NPs, mostly iron oxide including maghenite (γ-Fe₂O₃) and magnetite (Fe₃O₄), having ferro(i)magnetic or superparamagnetic property are widely applied for cell sorting/separation, MRI diagnosis, hyperthermia cancer therapy and drug targeting or delivery purpose in medical use. Superparamagnetic NPs, which can be attracted to and maintained at a precise location by an external magnet, have been of particular interest for numerous applications. The term magnetofection is referred to describe the action of a magnetic force on gene vectors combined with magnetic particles. These magnetic vectors have been exclusively based on iron oxide, for which there has been safety concern related to cytotoxicity. Although iron oxide NPs with cationic lipid or polymer transfection agents may result in higher expression level in vitro, there remain concerns regarding their safety profile. Moreover, an unfavorable “serum effect” on DNA-lipid transfection complex explains the dramatic reduction in transfection efficiency in vivo compared to in vitro culture with lipoplex transfection in serum-free medium. On the other hand, calcium phosphate NPs as non-viral vectors have been shown to have “appreciable serum tolerability” and demonstrate enhancement in transfection efficiency when administered in vivo.

The effect of calcium phosphate-based apatite coating layer has been developed for cell adhesion and growth enhancement (see US patent publications No. 2006/0024823). Additionally, the calcium hydroxyapatite (Hap) could utilize ion exchange to have multifunctional property (Japanese patent publication No. 2000327315). However, nano-sized material is difficult to fabricate through sintering process due to crystal growth and particle size enlargement. US patent publications No. 2007/0078520 showed the effect of the coverage of Hap layers, which could improve the biocompatibility, hindered the performance of central core materials even though it still maintained the fluorescence/magnetic property. In this invention, we use wet-chemical process instead of sintering method, and the delivery carrier/vehicle has been successfully developed to possess nano-range particle size, good biocompatibility, and efficient transfection behavior under low transfection dosage administration.

### SUMMARY OF THE INVENTION

In view of the above problems, the present invention provides safer and more effective superparamagnetic NPs in medical therapeutics and a manufacturing method thereof. In the present invention, deal with the problems in use of a conventional magnetic nanoparticles, to avoid presence of cytotoxicity by the magnetic nanoparticles on an organism, to package the magnetic nanoparticles by biocompatible medium preventing from the decrease of magnetic attraction effect of an applied magnetic field, to replace the metal ion located on a surface of the magnetic nanoparticles by other ions or molecules in the fabrication procedure, to simplify the process for manufacturing process compared to conventional magnetic NPs and high manufacturing cost, etc.

The present invention discloses a method for manufacturing superparamagnetic NPs in medical therapeutics, comprising the following steps: providing a suspension containing a number of calcium phosphate particles, and providing a metal ion solution containing a number of magnetic metal ions; then mixing the metal ion solution with the suspension to form a mixture; subsequently titrating the mixture with an alkaline solution so that the mixture having a basic pH; epitaxially growth of the number of magnetic metal ions on surfaces of the calcium phosphate particles, whereby a number of calcium phosphate particles having magnetic metal crystallites are formed; and finally isolating and collecting the calcium phosphate particles having the magnetic metal crystallites from the mixture. The calcium phosphate particles having the magnetic metal crystallites (mHap particles) possess superparamagnetic property.

The present invention discloses a superparamagnetic NPs, comprising at least one calcium phosphate particle as well as a number of magnetic metal crystallites, in which these magnetic metal crystallites are epitaxially bound to the surface of calcium phosphate particles inducing the mHap particles with superparamagnetic property.

The present invention also discloses an application of a superparamagnetic NPs made by the above method for manufacturing superparamagnetic NPs in medical therapeutics. Such superparamagnetic NPs in medical therapeutics are applied to the biomedical field in gene therapy, gene transfection, drug delivery, magnetic resonance imaging, tumor heat treatment, cell isolation and biosensors.

The present invention prompts by the need to develop magnetic NPs which could reduce the damage on cell in vitro or organisms in vivo and increase transfection efficiency under the attraction of a magnet. Currently, these magnetic vectors have been exclusively based on iron oxide, for which there has been concerns related to cytotoxicity. The core-shell structure of the outer layer such as polymer coating has been required to promote biocompatibility for bare iron oxide. The coverage could result in reducing magnetization, increasing process complexity and high cost. The problems have been solved in the present invention by well-controlled, wet-chemical methods at low temperature reaction for producing mHap nanoparticles that calcium phosphate crystallites can be rendered magnetic by the hetero-epitaxial growth of magnetite.

The case of hetero-epitaxy in which one crystalline phase, in this case magnetite, grows from the surface of a crystal of different composition. i.e., apatite. For hetero-epitaxy to occur the mismatch in the lattice parameters of the two crystals at the interfacial plane should be 15% or less. In the present invention, the mismatch in the magnetite and apatite lattice parameters are 12% (Hap a-axis versus magnetite a-axis) and 18% (Hap c-axis versus magnetite c-axis), close to the normally accepted criterion for hetero-epitaxy. Furthermore, the effectively control the particle size of the magnetite on Hap crystallite by hetero-epitaxy growth since the present invention manufactured method. This compositional difference along with the difference in crystallite size and shape may have resulted in a more favorable release of the plasmid DNA intracellularly and facilitated transport into the nucleus. Otherwise, the difference in transfection was overcome by the application of the magnetic field. The enhanced transfection under the action of the magnet may have been due to the effects of: 1) magnetic localization and retention of the NPs at the cell surface; 2) facilitated endocytosis or other process of passage of the NPs through the cell membrane; and/or 3) enhanced release of the plasmid intracellularly.

Calcium phosphate particles are stable at neutral pH and exhibit the principal form of calcium phosphate in the body—the mineral constituent of hard tissues. For example, Hap is favorable safety profile as a biomaterial based on its biocompatibility and biodegradability. While there are questions concerning the intrinsic biocompatibility of magnetic metallic NPs, the developed mHap particles which provide functionality to conventional calcium phosphate and improve biocompatibility for original iron oxide are suitable candidate as non-viral vectors for biomedical application.

The description on the content of the present invention above and the description on the embodiments below are used to exemplify and explain the principle of the present invention, and provide further explanation on the claims of the present invention.

### BRIEF DESCRIPTION OF THE DRAWINGS

The present invention will be illustrated in more detail as shown in the figures and description below, and thus are not limitative of the present invention, and wherein:

FIG. 1 is a flowchart of the manufacturing process of the Hap particles by the present invention.

FIG. 2 is a flowchart of the manufacturing process of the mHap particles by the present invention.

FIG. 3A is a crystalline structure analysis of the Hap particle determined using X-ray diffraction (XRD) patterns by the present invention. The identity of the peaks is based on reference profiles in the JCPDS for Hap [card #09-0432; Ca₅(PO₄)(OH)].

FIG. 3B is a crystalline structure analysis of the mHap particle determined using XRD patterns by the present invention. The identity of the peaks is based on reference profiles in the JCPDS for Hap [card #09-0432; Ca₅(PO₄)(OH)] and magnetite [card #19-0629; Fe₃O₄]. The arrows in the mHap pattern show the magnetite peaks.

FIG. 4A is a chemical composition analysis of the Hap particle including calcium, phosphorous, and iron content by the present invention determined by energy-dispersive X-ray spectroscopy analysis (EDX).

FIG. 4B is a chemical composition analysis of mHap particle including calcium, phosphorous, and iron content by the present invention determined by EDX.

FIG. 5 is a magnetization (M) loop versus applied magnetic field from the superconducting quantum interference device (SQUID) of the mHap particles measured at 300K by the present invention.

FIG. 6A is a morphology image of the Hap particles examined using transmission electron microscopy (TEM) by the present invention.

FIG. 6B is a morphology image of mHap particles examined using TEM by the present invention. The image of the taken is at the same magnification with FIG. 6A. The arrows show the magnetite crystallites in the mHap samples.

FIG. 6C is a high-resolution TEM (HRTEM) image of the mHap particles showing interplanar spacings corresponding to Hap by the present invention.

FIG. 6D is a HRTEM image of the mHap particles showing interplanar spacings corresponding to magnetite by the present invention.

FIG. 7 is a selected area electron diffraction (SAD) pattern of the mHap particles taken from a region comparable to that in FIG. 6B by the present invention. The arrows point out the 002 and 211 reflections from Hap and the 311 and 400 reflections from magnetite. The asterisk (*) and (§) show spots from the c-axis (002) of Hap aligned with one of the orthogonal directions of magnetite, consistent with heteroepitaxial growth.

FIG. 8A is an agarose gel showing binding affinity of Hap particles to plasmid DNA (pDNA) encoding glial cell line-derived neurotrophic factor (GDNF) sequence by the present invention. The prominent band is the pDNA remaining in solution after the removal of the Hap particles. Lanes 1-3 displayed results for the Hap NPs:pDNA weight ratios of 1600:1.5; 800:1.5; and 400:1.5. In lane 4, the pDNA was run as a control.

FIG. 8B is an agarose gel showing binding affinity of mHap particles to pDNA by the present invention. The prominent band is the pDNA remaining in solution after the removal of the mHap particles. Lanes 1-3 showed results for the mHap particles:pDNA weight ratios of 500:1.5; 250:1.5; and 125:1.5. In lane 4, the pDNA was run as a control.

FIG. 8C is an agarose gel showing binding affinity of magnetite to pDNA by the present invention. The prominent band is the pDNA remaining in solution after the removal of the magnetite, lanes 1-3 showed results for the magnetite:pDNA weight ratios of 500:1.5; 250:1.5; and 125:1.5. In lane 4, the pDNA was run as a control.

FIG. 9 is an enzyme-linked immunosorbent assay diagram showing GDNF experssion using mHap particles transfection with and without applied magnetic field for rat marrow-derived mesenchymal stem cells (MSCs) by the present invention. The mHap particles demonstrated overexpression of GDNF at each of the collection periods and displayed enhanced transfection efficiency under the action of a magnetic field.

### DETAILED DESCRIPTION OF THE INVENTION

The present invention relates to a method for biomaterials. The notable findings of the invention were that the superparamagnetic NPs in medical therapeutics comprising synthetic calcium phosphate crystallites could be modified to be made superparamagnetic property. And, the superparamagnetic NPs in medical therapeutics displayed enhanced gene transfection or drug delivery when used as non-viral vectors under the action of a magnetic field.

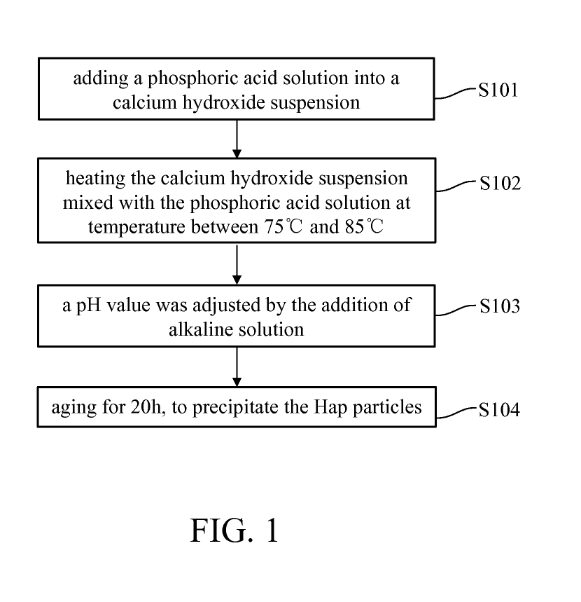

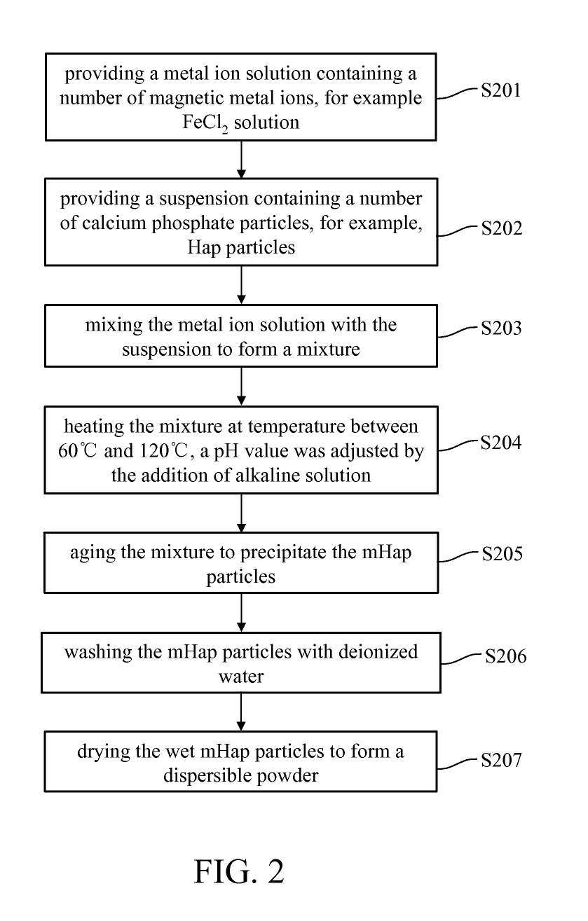

In an embodiment of the present invention, the manufacturing process for preformed calcium phosphate particles, as shown in FIG. 1, were treated with a solution of magnetic metal ions (i.e. metal ions solution) at a rate of 0.5˜3 mL/min followed by alkaline condition at 60˜120° C., as shown in FIG. 2, to form a superparamagnetic NPs. An average particle size of the calcium phosphate particles and the superparamagnetic NPs respectively are between 1˜600 nm, for example, between 1˜100 nm. The calcium phosphate particles can be prepared by experimental method, commercial product or replaced by apatite series including Hap, carbonate-fluorapatite, carbonate-hydroxylapatite, chlorapatite, fluorapatite, dicalcium phosphate, tricalcium phosphate and a mixture thereof. The ratio of calcium and phosphorous (Ca:P) was 1˜2.5. The metal ion solution is formulated by a compound of magnetic metal such as iron (Fe), cobalt (Co), chromium (Cr), nickel (Ni), gadolinium (Gd), magnesium (Mg), copper (Cu), manganese (Mn) and zinc (Zn) can provided by their compounds or derivatives. For example, ferric nitrate (Fe(NO₃)₃), ferric phosphate (FePO₄), ferric oxide (Fe₃O₄), cobalt oxide (CoO₂), gadolinium oxide (Gd₂O₃), ferric chloride (FeCl₃), ferrous chloride (FeCl₂) and cobalt nitride (CoN) aqueous solution can be supplied for the source of iron, cobalt or gadolinium ions. The metal ion solution contains a number of magnetic metal ions. In an embodiment of the present invention, the calcium phosphate particles are Hap particles and the metal ion solution is a FeCl₂ solution, and the present invention is not limited to this particular embodiment.

According to an embodiment of the present invention, the alkaline condition can be adjusted by an alkaline solution, for example, ammonium hydroxide (NH₄OH) solution, sodium hydroxide (NaOH) solution or sodium bicarbonate (NaHCO₃) solution. The process of the superparamagnetic NPs in medical therapeutics is formed for 2˜15 h (hour(s)) under alkaline condition and 60˜120° C.

Example for the superparamagnetic NPs manufacturing process described below is provided for illustration, and the present invention is not limited to this particular embodiment. Referring to FIG. 1, a phosphoric acid solution (ex. orthophosphoric acid solution) was added into a dispersed calcium hydroxide suspension (S101) at rate of 1.5 mL/min. The entire process was carried out at 75˜85° C. in a hot water bath (S102). For example, the entire process was carried out at 80° C. The final Ca:P ratio was 1.67 to match the stoichiometric ratio in Hap. The pH value was adjusted to 8 by the addition of NH₄OH during the precipitation process (S103), and aged for about 20 h (S104) to precipitate the Hap particles. The term, “nanoparticles” was adopted to describe the crystallite aggregates, which were from tens to hundreds of nanometers in diameter.

As shown in FIG. 2, providing the metal ion solution (S201) and providing a suspension containing a number of Hap particles (S202). Then, mixing the metal ion solution with the suspension at rate of 1.5 mL/min (S203) to form a mixture. The pH value of the mixture was adjusted to 8 by adding NH₄OH at 60˜120° C., for example 80° C. (S204), and the mixture aged for 2˜12 h (ex. 10 h) at 80° C. to precipitate the mHap particles (S205). The mHap particle is a Hap particle having a number of magnetic metal grains (or magnetite crystallites), i.e. the superparamagnetic NPs. Wherein the magnetic metal grains are epitaxially growing on surfaces of the Hap particle. Subsequently, the mHap particles are washed with deionized water three times (S206). Finally, the wet mHap particles were freezedried to form a dispersible powder (S207).

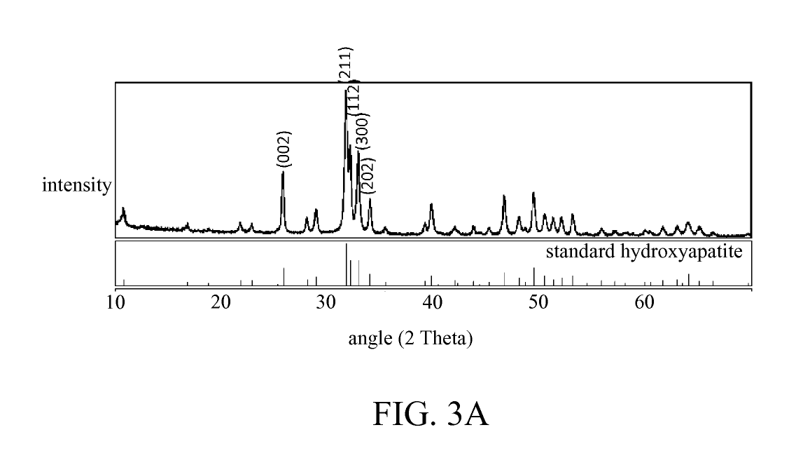

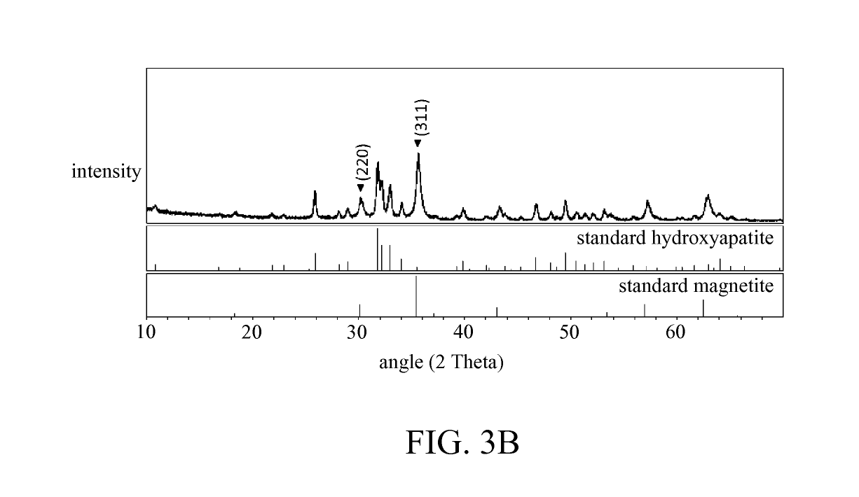

The present invention is that Hap was rendered superparamagnetic as the result of the formation of magnetite crystallites on the individual Hap crystallites. This is a case of hetero-epitaxy in which one crystalline phase, in this case magnetite, grows from the surface of a crystal of different composition, i.e., apatite. XRD confirmed the synthesized Hap structure of the crystallites comprising the referenced Hap particles (FIG. 3A). The diffraction peaks were broad indicating a smaller crystallite size in the Hap particles. The size of the apatite crystallites along the c crystallographic axis (c-axis) was estimated from line broadening to be 53 nm for Hap. These dimensions did not change significantly after the addition of the iron ions: 56 nm for mHap (FIG. 3B). The locations and relative intensities of the Hap diffraction peaks in the XRD patterns of mHap (FIG. 3B) were similar to those of the Hap samples (FIG. 3A) indicating that the apatite structure of the Hap particles was maintained, with no noticeable alteration of the apatite crystalline structure as a result of the addition of the iron ions. Of note in the XRD patterns from the mHap particles according to the present invention, compared to the nonmagnetic Hap samples, were the pronounced additional peaks at the 2 theta (θ) values of 30.18 and 35.48. These were identified as magnetite from the Joint Committee on Powder Diffraction Standards (JCPDS) card #19-0629 (FIG. 3B). The mHap (particles) powder was consistent with the presence of magnetite demonstrated by XRD.

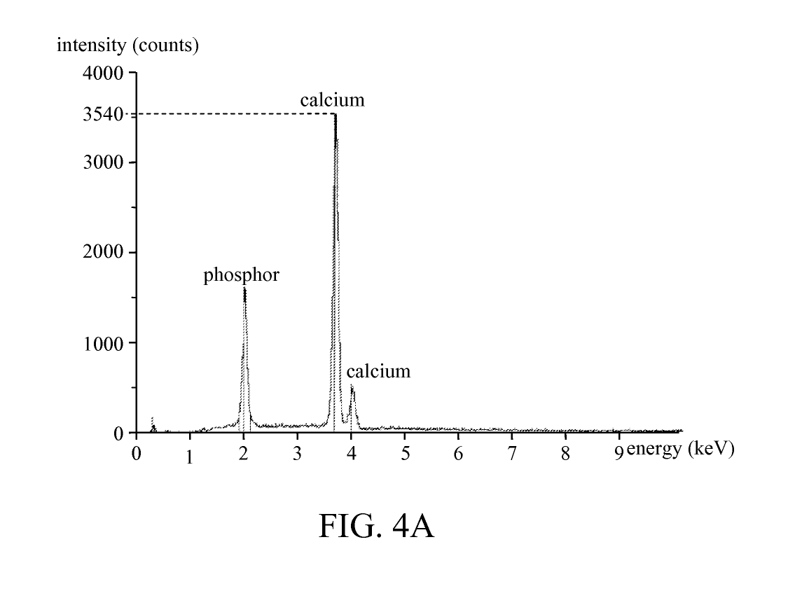

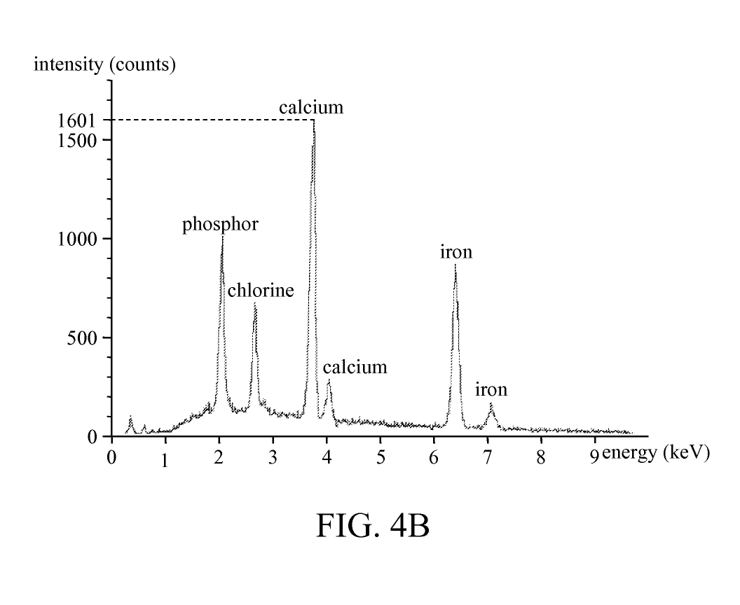

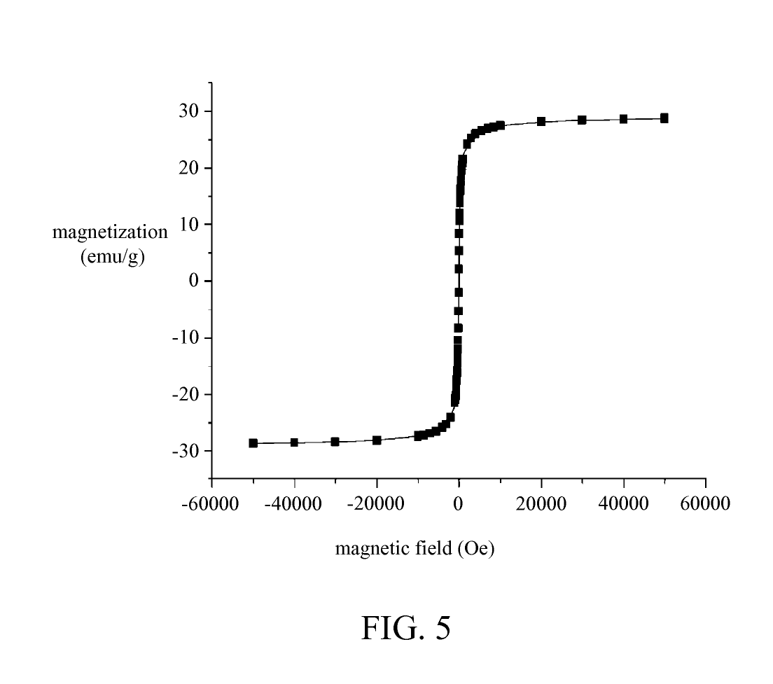

The chemical compositions of the Hap particles and mHap particles were determined by EDX analysis. There was no Fe detected in the Hap particles (FIG. 4A). After the addition of iron for the synthesis of the mHap particles, there was iron existence and only a slight drop in the Ca:P ratio for the mHap particles, indicating that most of the iron had not been substituted or ion-exchange for calcium (FIG. 4B). Moreover, virtually all of the mHap particles followed the movement of the magnet and could be extracted from the mixture using the magnet, leaving a clear solution. No such behavior was displayed by the Hap particles. Therefore, the synthesized mHap particles were not simple mixtures of the calcium phosphate particles and magnetite crystallites. Further, SQUID analysis demonstrated the magnetization behavior versus applied magnetic field for the mHap particles (FIG. 5). The absence of hysteresis in the curve of magnetization versus external magnetic field for mHap particles demonstrated that the mHap particles according to the present invention had no remanence and coercivity, reflecting the fact that there were no long-range magnetic dipole-dipole interactions among the mHap particles, and thus indicated superparamagnetic behavior. That the magnetization of mHap particles did not saturate at 50 KOe was also consistent with superparamagnetic behavior. The present invention is successfully prepared magnetized Hap particles during the process by iron addition which possessed superparamganetic property.

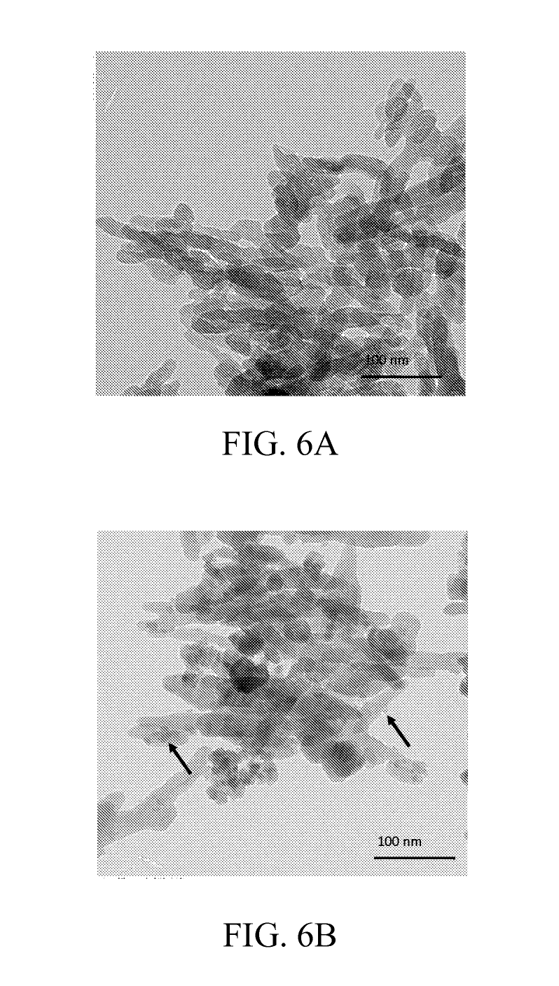

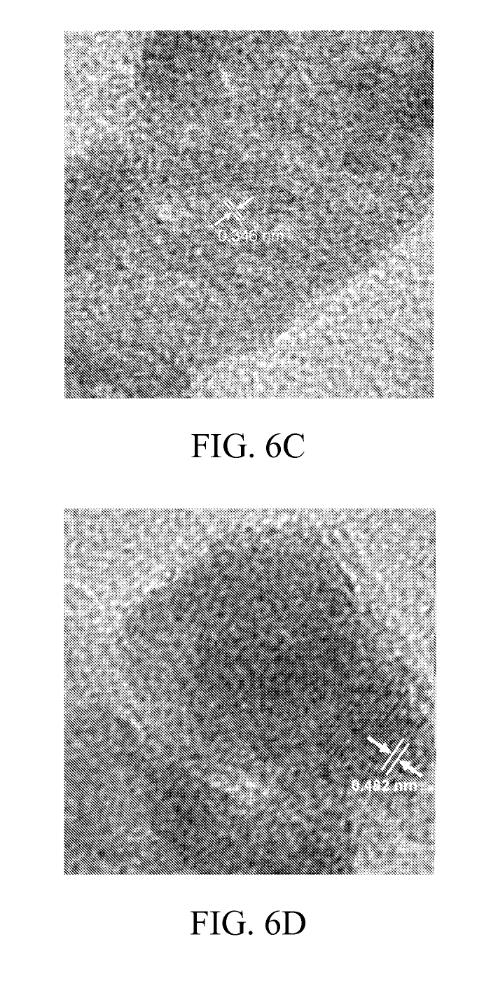

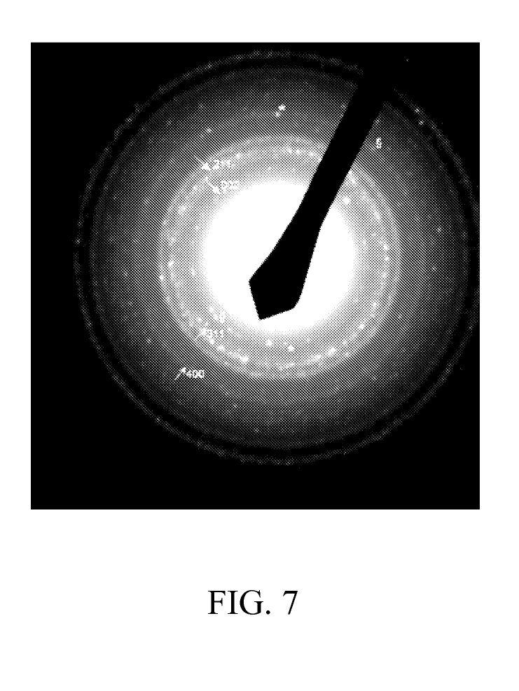

Furthermore, TEM revealed the clusters of crystallites making up the Hap particles which were from about 200-400 nm in diameter. The crystallites making up the Hap particles appeared in TEM in rod- and lath-like morphologies, 50-100 nm long by about 25 nm wide (FIG. 6A). Of note was the additional presence of small irregularly shapes discs, less than 10 nm in diameter, in the samples of the iron containing Hap particles (FIG. 6B). The Fourier images from high-resolution TEM of the mHap particles demonstrated planes with a spacing (0.346 nm) that corresponded with the c-axis of Hap, in the direction of the long axis of the crystallite (FIG. 6C). Other features in the high-resolution TEM demonstrated Fourier images with an interplanar spacing (0.482 nm) corresponding to magnetite (FIG. 6D). The results revealed crystallites consistent in size with magnetite (less than 10 nm in diameter) on the surfaces of the plate-like Hap crystallites. Additionally, SAD demonstrated the presence of magnetite along with Hap in the mHap samples, consistent with the XRD findings (FIG. 7). It was also interesting to find spots in the SAD pattern of mHap particles that corresponded with the principal orthogonal axis of magnetite aligned in the same direction as the c-axis of Hap particles (note the * and § in FIG. 6), also consistent with hetero-epitaxy. This invention was consistent with a coordinated alignment of the magnetite and Hap crystallites.

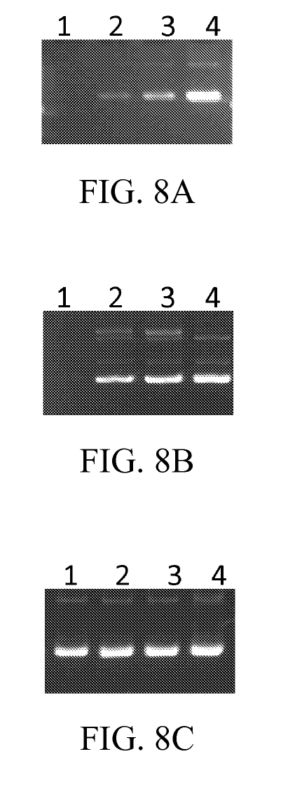

The goal of the current invention was to attempt to replace iron oxide for magnetofection with calcium phosphate particles rendered magnetic by the addition of iron ions. The present invention produced calcium phosphate particles with adherent magnetite crystallites. Moreover, according to the present invention, the mHap particles do provide improved pDNA binding and the benefits of the properties of the Hap particles, and importantly reduce the amount of iron oxide that would be introduced into the body. The Hap particles adsorbed virtually all of the pDNA from its solutions at the highest particles:pDNA (by weight, wt.) ratio (Table 1, FIG. 8A). As might have been expected, the percentage of pDNA bound by the samples increased with the ratio of particles:pDNA. Because the contribution of the iron to the weight of the mHap could not be established, direct comparison of the binding results for the Hap and magnetic samples (mHap and magnetite) could not be made in such away as to account for differences in particle number and surface area. However, the present invention estimated that the highest particles:pDNA wt. ratio for the mHap particles would be comparable with the highest particles:pDNA wt. ratio for the Hap particles (Table 1). Based on this comparison, the percentage of pDNA bound by the Hap particles remained about the same after addition of the iron ions to form the mHap particles (Table 1, FIG. 8B). In contrast, the magnetite samples displayed poor affinity for binding pDNA (FIG. 8C), compared to the Hap and mHap particles despite the fact that the small size of the magnetite crystallites would have indicated that the number of magnetite particles and their surface area would have been far greater than the mHap. The present invention revealed significant effect on mHap particles plasmid binding capacity.

TABLE 1
Percentage of pDNA binding to the particles.

| Mean ± | | | | | |
|---|---|---|---|---|---|
| (standard deviation) | Hap | | mHap | | Magnetite |
| sample particles: plasmid | 1600/1.5 | 97 ± 5 | 500/1.5 | 99 ± 1 | 8 ± 2 |
| DNA by weight ratio | 800/1.5 | 84 ± 13 | 250/1.5 | 46 ± 7 | 4 ± 2 |
| | 400/1.5 | 69 ± 5 | 125/1.5 | 19 ± 4 | 0 |

$Binding capacity = [ 1 - \frac{pDNA content ( residual )}{pDNA content ( initial )} ] \times 100 \%$

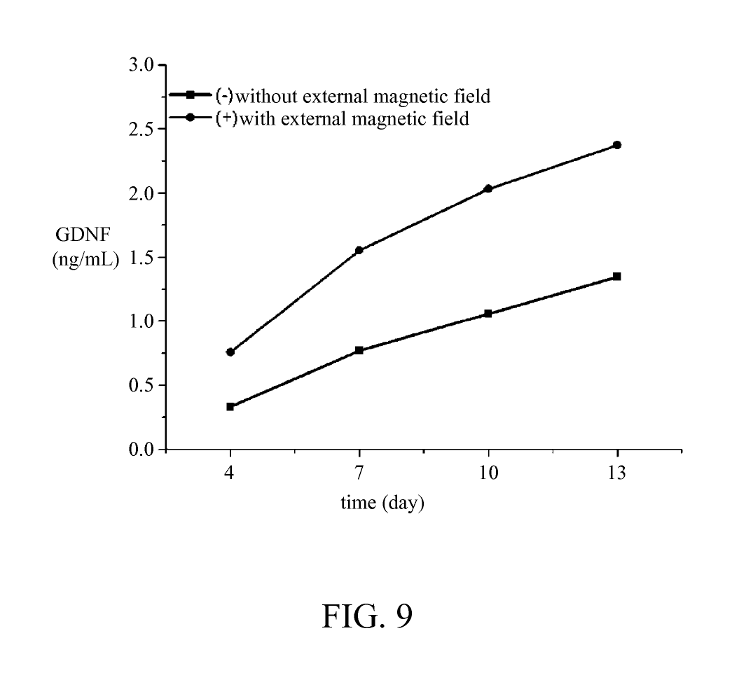

According to the present invention, the mHap particles showed substantial increases in the gene expression under an applied magnetic field, and were shown to be non-cytotoxic effect. Moreover, the amount of GDNF recovered in the medium approached therapeutic levels despite the small amount of plasmid delivered by the mHap particles. The mHap particle groups demonstrated overexpression and secretion of GDNF at each of the collection periods (FIG. 9). For mHap particles, the percentage increase in GDNF expression was significant enhanced at the first three collection periods. Magnetofection increased GDNF levels: 2.3 times at 4 days, 82% increase at 7 days, and 65% increase at 10 days (higher than non magnet controls). An approximate two fold increase in gene expression through 7 days post-transfection for the mHap particles resulting from the 15-min period under the influence of the magnetic field. Otherwise, application of the magnetic field increased the accumulated GDNF concentration in the mHap particle cultures from 1.4 to 2.4 ng/mL. The results demonstrated that the differences in GDNF levels were highly significant between groups with and without magnetofection. Considering the invention fact that the magnet was applied for only the first 15-min period that the cells were exposed to the pDNA-mHap particle complexes would enhance transfection. Also of note was that the LDH assay showed that there was no cytotoxicity of the mHap particles.

Besides, the two-week accumulated levels of GDNF of about 2 ng/mL in the magnetofection cultures with the mHap particles containing as little as 0.6 mg plasmid/well. Of importance is that the level of GDNF expression by MSCs following magnetofection was high enough to result in GDNF concentrations which were found to be close to therapeutic concentrations in previous in vitro investigations: 1 ng/mL increased by nearly 100% the number of trigeminal ganglion sensory neurons in culture at 5 days postplating; and 10 ng/mL nearly doubled dopamine neuron survival and reduced the rate of apoptosis from 6 to 3% in human embryonic dopamine neurons cultures.

The enhanced transfection under the action of the magnet may have been due to the effects of: 1) magnetic localization and retention of the mHap particles at the cell surface; 2) facilitated endocytosis or other process of passage of the mHap particles through the cell membrane; and/or 3) enhanced release of the plasmid intracellularly.

The present invention is further related to gene or drug delivery vehicle which preparation method described above for in vitro and in vivo delivery to achieve therapeutic purpose in clinic. Magnetofection using mHap particles according to the present invention will be capable of inducing expression of therapeutic doses locally. It will be helpful in future medical applications to retain the in vitro transfection efficiencies of the mHap particles in the presence of increasing amounts of serum supplementing the medium. Therefore, such effects will be important in providing an indication of the potential effectiveness of the non-viral vectors in vivo for clinical use; for example, for use in gene therapy, gene transfection, drug delivery, magnetic resonance imaging, tumor heat treatment, cell isolation or biosensors, and for use as a carrier for a biomolecule selected from the group consisting of nucleic acid, nucleotide, oligonucleotide, peptide, protein, antibody and lipid.

What is claimed is:

1. A method for manufacturing superparamagnetic nanoparticles in medical therapeutics, comprising the following steps:
providing a suspension comprising a number of calcium phosphate particles;
providing a metal ion solution comprising a number of magnetic metal ions;
mixing the metal ion solution with the suspension to form a mixture;
titrating the mixture with an alkaline solution so that the mixture has basic pH;
epitaxially growing the magnetic metal ions on surfaces of the calcium phosphate particles to form a number of calcium phosphate particles having a number of magnetic metal crystallites;
and isolating and collecting the calcium phosphate particles having the magnetic metal crystallites from the mixture, wherein the calcium phosphate particles having the magnetic metal crystallites possess superparamagnetic property.

2. The method for manufacturing superparamagnetic nanoparticles in medical therapeutics according to claim 1, wherein the alkaline solution is one of ammonium hydroxide solution, sodium hydroxide solution and sodium bicarbonate solution.

3. The method for manufacturing superparamagnetic nanoparticles in medical therapeutics according to claim 1, wherein the calcium phosphate particles comprising the magnetic metal crystallites have an average particle size between 1 nm and 600 nm.

4. The method for manufacturing superparamagnetic nanoparticles in medical therapeutics according to claim 3, wherein the calcium phosphate particles comprising the magnetic metal crystallites have an average particle size between 1 nm and 100 nm.

5. The method for manufacturing superparamagnetic nanoparticles in medical therapeutics according to claim 1, wherein the calcium phosphate particles are selected from hydroxyapatite particles, carbonate-fluorapatite particles, carbonate-hydroxylapatite particles, chlorapatite particles, fluorapatite particles, dicalcium phosphate particles, tricalcium phosphate particles and a mixture thereof.

6. The method for manufacturing superparamagnetic nanoparticles in medical therapeutics according to claim 1, wherein the material of the magnetic metal crystallites is selected from iron, cobalt, nickel, chromium, magnesium, zinc, copper, manganese and gadolinium.

7. The method for manufacturing superparamagnetic nanoparticles in medical therapeutics according to claim 1, further comprising heating the mixture to a temperature of 60° C. to 120° C. after the mixing of the metal ion solution with the suspension to form the mixture.

8. The method for manufacturing superparamagnetic nanoparticles in medical therapeutics according to claim 7, wherein the mixture is heated to a temperature of 80° C. to 85° C.

9. The method for manufacturing superparamagnetic nanoparticles in medical therapeutics according to claim 1, further comprising allowing the mixture to age for 2 h to 15 h after titrating the mixture with the alkaline solution so that the mixture has basic pH.

10. The method for manufacturing superparamagnetic nanoparticles in medical therapeutics according to claim 9, wherein the mixture is allowed to age for 10 h.

11. The method for manufacturing superparamagnetic nanoparticles in medical therapeutics according to claim 1, wherein the epitaxially growing of the magnetic metal ions on the surfaces of the calcium phosphate particles to form the number of calcium phosphate particles having the number of magnetic metal crystallites is carried out at a temperature of 60° C. to 120° C.

12. The method for manufacturing superparamagnetic nanoparticles in medical therapeutics according to claim 11, wherein the epitaxially growing of the magnetic metal ions on the surfaces of the calcium phosphate particles to form the number of calcium phosphate particles having the number of magnetic metal crystallites is carried out at a temperature of 80° C. to 85° C.

13. A calcium phosphate particle having magnetic metal crystallites made by the method for manufacturing superparamagnetic nanoparticles in medical therapeutics according to claim 1, for use in gene therapy, gene transfection, drug delivery, magnetic resonance imaging, tumor heat treatment, cell isolation or biosensors.

14. The calcium phosphate particle having magnetic metal crystallites made by the method for manufacturing superparamagnetic nanoparticles in medical therapeutics according to claim 13, for use as a carrier for a biomolecule selected from the group consisting of nucleic acid, nucleotide, oligonucleotide, peptide, protein, antibody and lipid.

15. A superparamagnetic nanoparticles in medical therapeutics, comprising:
at least one calcium phosphate particle; and
a number of magnetic metal crystallites, epitaxially bound to a surface of the calcium phosphate particle;
wherein the calcium phosphate particle having the magnetic metal crystallites possess superparamagnetic property.

16. The superparamagnetic nanoparticles in medical therapeutics according to claim 15, wherein the calcium phosphate particles are selected from hydroxyapatite particles, carbonate-fluorapatite particles, carbonate-hydroxylapatite particles, chlorapatite particles, fluorapatite particles, dicalcium phosphate particles, tricalcium phosphate and a mixture thereof.

17. The superparamagnetic nanoparticles in medical therapeutics according to claim 15, wherein the material of the magnetic metal crystallites is selected from iron, cobalt, nickel, chromium, magnesium, zinc, copper, manganese and gadolinium.

18. The superparamagnetic nanoparticles in medical therapeutics according to claim 15, wherein the calcium phosphate particle comprising the magnetic metal crystallites has a particle size between 1 nm and 600 nm.

19. The superparamagnetic nanoparticles in medical therapeutics according to claim 18, wherein the calcium phosphate particle comprising the magnetic metal crystallites has a particle size between 1 nm and 100 nm.

20. The superparamagnetic nanoparticles in medical therapeutics according to claim 15, wherein the calcium phosphate particle having the magnetic metal crystallites is used in gene therapy, gene transfection, drug delivery, magnetic resonance imaging, tumor heat treatment, cell isolation or biosensors, and used as a carrier for a biomolecule selected from the group consisting of nucleic acid, nucleotide, oligonucleotide, peptide, protein, antibody and lipid.
